(19) United States
(12) Patent Application Publication
(10) Pub. No.: US 2024/0260647 A1
(43) Pub. Date: Aug. 8, 2024

(54) FORMULATION OF MICROPARTICLES BASED ON POLYPHENOLIC COMPOUNDS CAPABLE OF SCAVENGING FREE RADICALS PRESENT IN POLLUTED AIR AND IN SMOKE

(71) Applicant: Iman Emami, Paris (FR)

(72) Inventor: Iman Emami, Paris (FR)

(21) Appl. No.: 18/563,952

(22) PCT Filed: May 24, 2022
(86) PCT No.: PCT/FR2022/050990

§ 371 Date: Nov. 24, 2023

(30) Foreign Application Priority Data
May 24, 2021 (FR) FR2105378

(51) Int. Cl. A24D 3/14; A24D 3/02; A24D 3/04; A24D 3/06; A41D 13/11; A61K 9/16; A61K 31/05; A61K 47/10

(52) U.S. Cl. CPC A24D 3/14; A24D 3/0216; A24D 3/048; A24D 3/061; A41D 13/11; A61K 9/16; A61K 31/05; A61K 47/10

(57) ABSTRACT

The disclosure relates to the field of free radical scavengers. More particularly, the disclosure concerns microparticles associated with polyphenolic compounds capable of capturing free radicals present in polluted air or smoke, in particular tobacco and/or cannabis-based cigarette smoke. The disclosure also relates to a process for preparing a cigarette filter and the filter obtained, as well as a process for preparing a filter fabric and the fabric obtained.

The invention relates to the field of free radical scavengers. More particularly, the invention concerns microparticles associated with polyphenolic compounds capable of capturing free radicals present in polluted air or smoke, in particular tobacco, CBD and/or cannabis-based cigarette smoke or mixtures thereof

### FIELD OF THE INVENTION

The present invention relates to the use of polyphenolic compounds or their derivatives as free radical scavengers in devices designed to reduce the harmful effects of air pollution, smoke generated by the combustion of tobacco, smoke generated by the combustion of cannabis (medical cannabis is considered here), or smoke generated by the combustion of CBD or a mixture of these smokes, or a mixture of these smokes and air pollution. Such a use is described in patent EP1041899B.

Many polyphenolic compounds are known for their beneficial properties in areas as varied as hypertension, hypercholesterolemia, cardiovascular disease, viral infections and inflammatory phenomena. The anti-lipoperoxidant and anti-carcinogenic activities of certain polyphenols have also been described.

In particular, epigallocatechin-3-gallate (EGCG), the main polyphenol present in green tea, has been shown to possess potent anti-inflammatory and antiproliferative activity, capable of selectively inhibiting cell growth and inducing apoptosis in cancer cells without harming normal cells (D N Syed et al. Green tea polyphenol EGCG suppresses cigarette smoke condensate-induced NF-κB activation in normal human bronchial epithelial cells Oncogene Volume 26, pages 673-682 (2007) doi.org/10.1038/sj.onc.1209829).

However, to date, the use of polyphenolic compounds as a means of capturing the free radicals present in cigarette smoke and more generally in the air, and liable to be inhaled, has been little studied and needs to be improved if it is to be exploitable at industrial level.

### DESCRIPTION OF THE INVENTION

The authors of the present invention have now discovered that incorporating polyphenols or their derivatives into microparticles effectively eliminates free radicals from cytotoxic molecules in smoke from tobacco, cannabis (for medical purposes), CBD, air pollution or mixtures thereof. These microparticles are incorporated into a cigarette filter or onto a non-woven fabric (meltblown extruded type). The mixture of polyphenolic compounds formulated in microparticles enables free radicals to be selectively absorbed from the gas phase and the semi-liquid phase (tars) as they pass through the filter or filter fabric.

The present invention therefore concerns the use of polyphenolic compounds formulated in microparticles as free radical scavengers.

In particular, it concerns a formulation of microparticles associated with polyphenolic compounds and their derivatives, notably in the form of a rosemary extract, in which the microparticles consist of a mixture of 2 to 4 saturated vegetable oils whose melting temperatures differ by at least 10° C.

This formulation can be used as a free-radical scavenger in tobacco cigarette filters, CBD cigarette filters, cannabis cigarette filters, or cigarette filters based on their mixture and air filter fabric, particularly in a mask.

The invention also relates to a process for preparing a cigarette filter and the filter obtained, as well as a process for preparing a filter fabric and the fabric obtained.

### Advantages of the Invention

The formulation of polyphenolic compounds according to the invention has two main advantages.

Firstly, this formulation, based on at least 2 vegetable oils, creates a surface effect which optimizes the anti-free radical efficacy of the polyphenolic compounds. Once the oil/polyphenolic compound mixture has been heated and cooled, the polyphenolic compounds are pushed to the outside of the particle, exposing them to contact with the air, where they can interact with free radicals. These phenolic compounds capture free radicals, in particular OH^{o} free radicals.

This composition has a neutralizing effect on the effect of tars due to the contacts established on the particle surface. In the case of cigarette smoke, tars compete with phenolic compounds to interact with free radicals. These free radicals in the semi-liquid phase are also trapped by polyphenolic compounds like those in the gas phase; this effect has been demonstrated in particular with a rosemary extract rich in polyphenols.

On the other hand, this approach provides particles of a size that avoids the risk of ingestion, whether of the particles present in the filter or on the filter fabric. To this end, particle size is adapted to the chosen application. Particles must be large enough to be retained on their support (filter or fabric), while taking into account constraints linked to product preparation and temperature of use.

Cigarette filters can be used for tobacco and/or cannabis consumption, notably for therapeutic purposes, or for CBD to capture free radicals and thus counter their neurodegenerative effects.

Filter fabrics are particularly suitable for making anti-pollution masks that can be used in any environment polluted by fine particles such as tar, especially for asthmatics, cyclists and sportsmen in urban environments.

### DETAILED DESCRIPTION OF THE INVENTION

A first object of the invention concerns the formulation of microparticles associated with polyphenolic compounds and their derivatives, in particular in the form of a rosemary extract, in which the microparticles consist of a mixture of 2 to 4 saturated vegetable oils whose melting temperatures differ by at least 10° C.

By “derivatives” we mean in particular compounds derived from polyphenolic compounds by substitution of the hydrogen atom of at least one of the hydroxy groups of the polyphenolic compounds by a C1-C6 alkyl group, or a (C1-C4 alkyl) carbonyl group. Acetates such as carnosic acid acetates and rosmarinic acid acetates are preferred derivatives of the polyphenolic compounds used according to the invention.

On the other hand, derivatives of polyphenols used in accordance with the invention such as carnosol, rosmanol, rosmarinic acid, carnosic acid may correspond to isomers of said polyphenols such as in particular epirosmanol and isorosmanol (Nakatani et al., Agric. Biol. Chem. 1984, vol. 48, no 8, pp 2081-2085, https://doi.org/10.1080/00021369.1984.10866436).

These compounds can be obtained by conventional chemical synthesis or by biotechnological means, using processes known to those skilled in the art. They can also be isolated from plant extracts.

In a preferred embodiment of the invention, the polyphenols used are provided in the form of an extract of rosemary (Rosmarinus officinalis L.). Such a plant extract can be obtained by extraction with a polar solvent such as an alcoholic or hydroalcoholic solvent. The alcohol used as solvent can be ethanol in particular. This extract can also advantageously be obtained using supercritical carbon dioxide and is then richer in polyphenolic compounds. Such a plant extract can be obtained by extraction with a polar solvent, an apolar solvent, a mixture of polar and apolar solvents, using supercritical carbon dioxide or the successive use of all these methods.

Preferably, the plant extract used according to the invention is obtained by polar solvent extraction followed by apolar solvent extraction followed by supercritical CO2 extraction.

Rosemary extraction is preferably carried out on dried plants, for example on rosemary branches, cut and sun-dried for 4 to 5 days, or on distillation residues low in essential oil content.

Polyphenolic compounds or their derivatives, obtained by chemical synthesis, by biotechnological means, or by extraction from plants, can be used alone or in mixtures in accordance with the invention.

Polyphenolic compounds or their derivatives can be used in free form, or they can be conjugated or coupled to a carrier to promote and amplify the absorbent action of the polyphenol-carrier combination.

By “saturated vegetable oil” is meant any saturated vegetable oil known to the person skilled in the art, and in particular hydrogenated palm oil, hydrogenated castor oil and cutina, etc. . . . .

In a particular embodiment, the polyphenolic compounds are selected from carnosol, rosmanol, rosmarinic acid, carnosic acid and derivatives thereof.

In another particular embodiment, the polyphenolic compounds consist of a mixture of carnosol, carnosic acid and rosmarinic acid or derivatives thereof.

In another particular embodiment, the polyphenolic compounds consist of a mixture composed wholly or partly of carnosol.

In another particular embodiment, the polyphenolic compounds are constituted by a mixture composed in whole or in part of carnosic acid.

In a preferred embodiment of the invention, said polyphenolic compounds are provided by a rosemary extract purified to at least 60%.

The weight ratio between the polyphenolic compounds and the oil mixture is between 25:75 and 65:35.

In the context of the present invention, the difference in melting temperature between the oils is at least 10° C., and may be at least 15° C.

A second object of the invention concerns the use of a formulation as defined above as a free radical scavenger in tobacco cigarette filters, CBD cigarette filters, cannabis cigarette filters, or cigarette filters based on a mixture thereof and in an air filter fabric, particularly in a mask.

Microparticles behave in an “ingenious” way. Indeed, they present a multiplied contact surface for the oxidation-reduction reaction, which contributes to the elimination of free radicals. By increasing the free enthalpy of the reaction, the adduction of gas-phase radicals to microparticles containing polyphenolic compounds is accelerated, rather than their adduction to tar particles. The result is a 30% to 65% reduction in tar radicals (semi-liquid phase), particularly semi-quinones, making tars less oxidizing and less mutagenic. This can be described as a tar-neutralizing effect.

In this way, the microparticles reduce the mutagenic and carcinogenic effect of the smoke and slow down the stiffening effect that takes place in the arteries due to the presence of oxidizing compounds.

A third object of the invention concerns a process for preparing a cigarette filter capable of capturing free radicals, in particular the free radicals present in smoke, consisting in:
mixing microparticles formed from a mixture of 2 to 4 saturated vegetable oils, whose melting temperatures differ by at least 10° C., with polyphenolic compounds or their derivatives, in particular in the form of a purified rosemary extract, and
incorporating said microparticles in a filter for tobacco cigarettes, filter for CBD cigarettes, filter for cannabis cigarettes or filter for cigarettes with a mixing density of 0.3 mg/mm3 to 1 mg/mm3.

A fourth object of the invention relates to a cigarette filter obtained by the process described above in which the microparticles have a size of between 70-200 microns and the filter obtained has a pressure drop of less than 1200 mm of water.

In a particular embodiment, the microparticles are incorporated into the part of the filter located on the side of the substance to be consumed over a length of 3 mm to 10 mm. Preferably, the part of the filter containing the microparticles is located in a 6 mm-long zone, 3 mm from the inner end of the filter (on the tobacco or other substance to be consumed side). This arrangement prevents the microparticles from coming into direct contact with excessive heat, which would cause them to melt, and also mechanically retains the microparticles so that they do not migrate towards the mouth, preventing them from being swallowed.

The use of such a filter leads to a reduction in free radicals in the gas phase, and a reduction in free radicals in the semi-liquid phase, resulting in a reduction in oxidative stress for smokers, a reduction in the oxidative effect of smoke, a reduction in inflammatory effects, a reduction in accelerated ageing, and a reduction in the risk of exposure to diseases: stroke, neurodegeneration, heart attack, cancer, etc. . . . .

A fifth object of the invention concerns a process for preparing a filter fabric capable of capturing free radicals, in particular the free radicals present in smoke and polluted air, consisting of:
mixing microparticles formed from mixtures of 2 to 4 saturated vegetable oils, whose melting temperatures differ by at least 10° C., with polyphenolic compounds or their derivatives, in particular in the form of a purified rosemary extract, and
deposit these microparticles on the surface of a filter fabric (of the meltblown extruded type) with a density of 15 gsm (gram per square meter) to 80 gsm.

A sixth object of the invention concerns a filter fabric obtained by the process described above, in which said microparticles have a size of between 1 and 20 microns and a pressure drop less than 150% of the FFP2, FFP3 or N95 standard pressure drop.

This filter fabric is particularly suited to the preparation of a mask to capture free radicals present in polluted air. In this case, the microparticles are applied to the outer face of at least one of the meltblown layers, preferably at least to the outermost meltblown layer of the mask. This type of mask is particularly suitable for asthmatics and athletes, or for use in polluted environments such as urban environments.

The absorption of free radicals and the induced reduction in the impact of cytotoxic molecules (see Alexandrov, K., Rojas, M., & Rolando, C. (2006). DNA damage by benzo (a) pyrene in human cells is increased by cigarette smoke and decreased by a filter containing rosemary extract, which lowers free radicals. Cancer research, 66(24), 11938-11945. DOI: 10.1158/0008-5472. CAN-06-3277) reduce oxidative stress, reduce cardiovascular disease, reduce the rate of mutagenesis in cellular tissues, reduce lipid oxidation, reduce neurodegenerative disease, reduce the risk of cardiovascular accidents, reduce the frequency of asthma attacks, particularly during pollution peaks, reducing the risk of cancer in smokers, particularly lung and lower tract cancer, reducing the risk of testicular cancer, reducing inflammation of the mouth, reducing inflammation of the tongue and upper tract, and reducing inflammatory phenomena.

The present invention will be better understood on reading the following examples, which are provided by way of illustration and are in no way to be considered as limiting the scope of the present invention.

### DESCRIPTION OF FIGURES

FIG. 1: Schematic view of the process for preparing a filter containing rosemary extract.

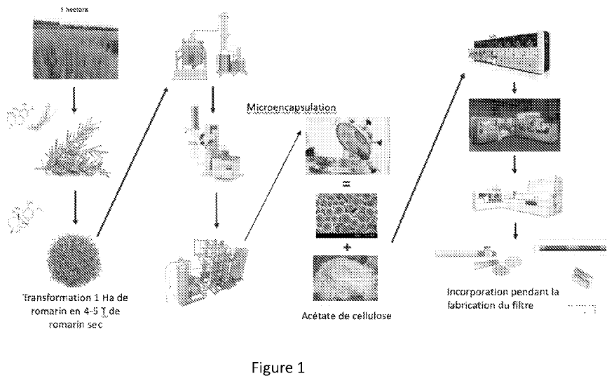

### EXAMPLES

### Example 1: Preparation of a Rosemary Extract

Rosemary (Rosmarinus officinalis L.) spikes are extracted with ethanol at 65° C. The volume of ethanol used (in liters) corresponds to five times the weight in kg of the rosemary spikes.

The extract is then purified and enriched in polyphenols by selective extraction with supercritical CO2. Depending on the temperature setting (between 40° C. and 100° C.) and the pressure (between 1 and 170 bar), the extract is purified and selectively enriched in its various components to achieve a carnosic acid and caronsol purity of over 50%.

Washing with one or more solvents significantly reduces the odor of the extract to 50%, while increasing the concentration of carnosic acid and caronsol to 65% or more.

Such an extract contains the following compounds: carnosol, rosmanol, rosmadial, carnosic acid, genkwanine, rosmarinic acid . . . .

The proportions of these various components vary according to the rosemary plant used. Generally speaking, an extract is obtained comprising around 10% to 25% rosmarinic acid, around 40% to 50% carnosic acid and around 5% to 10% carnosol.

### Example 2: Incorporating the Mixture into a Cigarette Filter or Filter Cloth

A cigarette filter is prepared by incorporating microparticles onto the surface of cellulose acetate or into crepe paper as it is wound. Preferably, the part of the filter containing the microparticles is located in a 6 mm-long zone, 3 mm from the inner end of the filter (on the tobacco or other substance to be consumed side).

The filter fabric is prepared by spreading the particles over the fabric surface and electrostatically charging them using a process known to the skilled person.

### Example 3: Method for Evaluating the Effectiveness of the Filter or Fabric Containing the Mixture of Microparticles and Nanoparticles and Polyphenols and their Derivatives

### 1. Computer-Aided Modelling

The effectiveness of the cigarette filter or filter fabric in capturing the free radicals thus prepared is first demonstrated by computer-aided modelling, using the Monte Carlo method, which calculates the number of encounters between a carcinogenic target molecule and a polyphenolic compound used in accordance with the invention.

The quantity of free-radical cytotoxic molecules present in cigarette smoke was calculated on both sides of the filter.

The quantity of free-radical cytotoxic molecules present in the air passing through the filter fabric was calculated on both sides per cm2 of filter fabric surface.

The number of cytotoxic molecules after filtration is a function of the volume of smoke passing through the filter, the surface area of the filter, the concentration of cytotoxic molecules in the smoke and the concentration of particles derived from a mixture of saturated vegetable oils and polyphenols in the filter.

The authors of the present invention have shown that 10 mg of particles in a cigarette filter reduces the level of cytotoxic free radical molecules in tobacco, CBD or cannabis cigarette smoke by over 60%.

### 2. Quantitative Mass Spectrometry Determination of Free Radical Filtration in Cigarette Smoke

The quantitative free radical filtration mass spectrometry assay is carried out using liquid chromatography (LC) and tandem mass spectrometry (LC/MS-MS) in multiple reaction monitoring (MRM) mode to quantify the OH^{o} free radical content of cigarette smoke. This mode provides a more specific and sensitive analysis. Indeed, the spectrometer detects only a few transitions rather than an entire mass spectrum, resulting in a higher signal-to-noise ratio. The 4,5,5-trimethyl-pyrroline-N-oxide (TMPO) spin trap was used in benzene. The experiment was performed by specifying the mass of the parent ion and monitoring its transition. A commercially available spin trap, 3-amino-2,2,5,5-tetramethyl-1-pyrrolidinyloxide, was used as an internal standard for free radical quantification. The absolute amount of free radicals measured here was in good agreement with the ESR experiments.

The results are presented in Table 1 below.

TABLE 1
Comparative results of quantitative mass spectrometry assay of cigarette
smoke free radical filtration by incorporated dual filters: the
first reference is additive-free. Filter A contains 15 mg of rosemary
extract distributed over a length of 6 mm. Filter B contains 15
mg of particles from the above-mentioned mixtures of rosemary extract
and saturated vegetable oils, spread over a length of 6 mm. All
three filters are based on cellulose acetate.

| | Reference | Filter A | Filter B |
|---|---|---|---|
| 6 mm deposit and filter length | 6.27 | 6.27 | 6.27 |
| Added rosemary extract, | — | 15 | 15 |
| or particles by weight | | | |
| (mg) | | | |
| Pressure drop (mm H₂O) | | 86 | 86 |
| Puffs for 5 cigarettes | 72 | 74 | 74 |
| Numbre of free radicals | 9.2 | 6.8 | 1.5 |
| OH° × 10⁻¹⁵ | | | |
| Free radicals scavenging | — | 26 | 85 |
| (%) | | | |

### 3. Quantitative Mass Spectrometric Determination of Free Radical Filtration Through a Filter Fabric

To test the anti-free radical effect associated with incorporating microparticles according to the invention into a filter fabric, 25 gsm of 5-micron microparticles were deposited on the outer surface of a meltblown.

The analysis methodology used was the same as that described above for filters.

The results show that the presence of microparticles enables at least 70% of OH^{o} free radicals to be captured.

1. Formulation of microparticles associated with polyphenolic compounds and/or their derivatives, especially in the form of rosemary extract, wherein the microparticles consist of a mixture of 2 to 4 saturated vegetable oils whose melting temperatures differ by at least 10° C.

2. Formulation according to claim 1 wherein said polyphenolic compounds are selected from carnosol, rosmanol, rosmarinic acid, carnosic acid, and derivatives thereof.

3. A formulation according to claim 1 wherein said polyphenolic compounds consist of a mixture of carnosol, carnosic acid and rosmarinic acid and/or derivatives thereof.

4. A formulation according to claim 1 wherein said polyphenolic compounds consist of a mixture composed wholly or partly of carnosol.

5. A formulation according to claim 1 wherein said polyphenolic compounds consist of a mixture composed wholly or partly of carnosic acid.

6. A formulation according to claim 1 in which said polyphenolic compounds are provided by a rosemary extract purified to at least 60%.

7. Use of a formulation as defined in claim 1 as a free radical scavenger in tobacco-based cigarette filters, CBD-based cigarette filters, cannabis-based cigarette filters, or cigarette filters based on a mixture thereof and in an air filter fabric, in particular in a mask.

8. A method of preparing a cigarette filter capable of capturing free radicals, in particular free radicals present in smoke, consisting in:
mixing microparticles formed from a mixture of 2 to 4 saturated vegetable oils, whose melting temperatures differ by at least 10° C., with polyphenolic compounds or their derivatives, in particular in the form of a purified rosemary extract, and
incorporate these microparticles in a filter for tobacco cigarettes, filter for CBD cigarettes, filter for cannabis cigarettes or filter for cigarettes with a mixture density of 0.3 mg/mm3 to 1 mg/mm3.

9. Cigarette filter obtained by the process according to claim 8 wherein said microparticles have a size between 70-200 microns and the filter has a pressure drop of less than 1200 mm of water.

10. Process for preparing a filter fabric capable of capturing free radicals, in particular free radicals present in smoke and polluted air, consisting of:
mixing microparticles formed from a mixture of 2 to 4 saturated vegetable oils, whose melting temperatures differ by at least 10° C., with polyphenolic compounds or their derivatives, in particular in the form of a purified rosemary extract, and
deposit these microparticles on the surface of a filter fabric with a density of 15 gsm to 80 gsm.

11. Filter fabric obtained by the process according to claim 10 wherein said microparticles have a size of between 1 and 20 microns and a draft resistance of less than 150% of the FFP2 or N95 or FFP3 standard.
